(19) United States
(12) Patent Application Publication
(10) Pub. No.: US 2023/0404391 A1
(43) Pub. Date: Dec. 21, 2023

(54) REFINED PROCEDURE FOR OPHTHALMIC ABERRATION MEASUREMENT

(71) Applicant: iCRx, Inc., Tucson, AZ (US)

(72) Inventors: Pierre A. Blanche, Tucson, AZ (US); Erol Ozgur, Tucson, AZ (US); Ram Voorakaranam, Tucson, AZ (US)

(21) Appl. No.: 17/843,653

(22) Filed: Jun. 17, 2022

(51) Int. Cl. A61B 3/103

(52) U.S. Cl. CPC A61B 3/103

(57) ABSTRACT

The present invention is directed to methods and devices for ophthalmic aberration measurement using a refractometer and phoropter. The present invention features a method for improving accuracy of measurements from an auto-refractometer to provide an optimal prescription for a patient's vision. The method may comprise measuring, by the auto-refractometer, a measurement of the patient's vision in an eye and applying, by an auto-phoropter operatively coupled to the auto-refractometer, a correction within a line of sight between the eye and the auto-refractometer. The correction may comprise an increase in visual acuity based on a correction factor. The method may further comprise repeating these steps until the patient's vision has been optimally corrected.

### FIELD OF THE INVENTION

The present invention is directed to methods and devices for ophthalmic aberration measurement using a refractometer and phoropter.

### BACKGROUND OF THE INVENTION

The automated ophthalmic aberration measurement by auto-refractometer uses some sort of wavefront sensor, such as Shack-Hartmann sensor, to determine the error imparted to the light reflected back by the subject retina. In such a measurement, there is an inherent trade-off between range of measurement (in diopter), and measurement accuracy. This is because of limitations in the sensor size (pixel count), and resolution (pixel pitch). High accuracy requires high pixel count, and high range of diopter requires high pixel count. For a real sensor (CCD/CMOS), both these metrics are finite. There are also limitations related to the human factors. Some people have difficulty to align their eyes to the optical axis of the system, particularly when they have a high amount of aberration. An impaired alignment causes errors in the measurements.

In an auto-phoropter, the subject eye aberration is compensated by some refractive optics (e.g. tunable lenses) such that vision is corrected or improved. Auto-refractometer and auto-phoropter can be combined in a single machine such that the measurement of the eye aberration is performed at first and then the correction is automatically presented to the subject so he/she can appreciate the proposed correction. The correction can be performed using a set of fluidic lenses that has the capacity to correct for spherical and cylindrical aberrations.

For subjects with large ophthalmic aberrations (such as >3 diopters), the measurement accuracy of the auto-refractometer can be limited because the wavefront arriving at the sensor has large distortions. When knowing the subject's actual prescription, or after a first measurement has been acquired, it is possible to compensate for the distortion by using the fluidic lenses of the auto-phoropter function of the instrument. However, this procedure proves to give over-corrected results when a second (or more) measurement is performed by the auto-refractometer. This over-correction measurement is only observed with human subjects and not with a model eye. The latter always gives accurate measurement.

The over-correction can be explained by the fact that human subjects have a tendency to accommodate when the vision is restored or close enough. This accommodation increases the eye refractive power and the measurement is over-corrected compared to far-vision. Since the accommodation reflex cannot be controlled and depends on the individual, it cannot be subtracted from the measurement.

So, it is difficult or even impossible to improve the accuracy of a refractive measurement by taking successive measurements where the aberration is corrected based on the previous measurement. This often leads to diverging refractive numbers. Thus, there exists a present need for a refined method for automated ophthalmic aberration measurement for use with a standard auto-refractometer and auto-phoropter.

### BRIEF SUMMARY OF THE INVENTION

It is an objective of the present invention to provide methods and devices that allow for improving accuracy of measurements from an auto-refractometer to provide an optimal prescription for a patient's vision, as specified in the independent claims. Embodiments of the invention are given in the dependent claims. Embodiments of the present invention can be freely combined with each other if they are not mutually exclusive.

The present invention features a method for improving accuracy of measurements from an auto-refractometer to provide an optimal prescription for a patient's vision. The method may comprise measuring, by the auto-refractometer, a measurement of the patient's vision in an eye and applying, by an auto-phoropter operatively coupled to the auto-refractometer, a correction within a line of sight between the eye and the auto-refractometer. The correction may comprise an increase in visual acuity based on a correction factor. The method may further comprise repeating these steps until the patient's vision has been optimally corrected.

The present invention features a system for improving accuracy of measurements from an auto-refractometer to provide an optimal prescription for a patient's vision. In some embodiments, the system may comprise the auto-refractometer, an auto-phoropter operatively coupled to the auto-refractometer, and a computing device communicatively coupled to the auto-refractometer and the auto-phoropter. The computing device may be capable of measuring, by the auto-refractometer, a measurement of the patient's vision in an eye and applying, by an auto-phoropter operatively coupled to the auto-refractometer, a correction within a line of sight between the eye and the auto-refractometer. The correction may comprise an increase in visual acuity based on a correction factor. The computing device may further be capable of repeating these steps until the patient's vision has been optimally corrected.

One of the unique and inventive technical features of the present invention is the measurement of a patient's prescription through use of an auto-refractometer by applying a fractional correction via an auto-phoropter. Without wishing to limit the invention to any theory or mechanism, it is believed that the technical feature of the present invention advantageously provides for a superior method of eyesight prescription generation compared to prior methods that apply an entire correction at once. None of the presently known prior references or work has the unique inventive technical feature of the present invention.

Here, another inventive aspect of the present invention is the capability to perform an objective refinement procedure for the patient. This refinement measurement does not require any feedback from the patient, in a similar fashion to the initial measurement. The present invention guarantees the objective nature of the measurement is preserved, while maintaining a better accuracy in the assessment of the refractive error.

Furthermore, the prior references teach away from the present invention. For example, prior references teach that a first objective measurement should be followed by a subjective evaluation in order to find the most suitable prescription. On the contrary, the present invention features an objective measurement, followed by an additional, fine-tuned objective measurement that requires no feedback from the patient. The adjustment to prevent accommodation guarantees the success of the objective measurement. Thus, the prior references teach away from the inventive feature of the presently claimed invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1 shows a flow chart of a method for improving accuracy of measurements from an auto-refractometer to provide an optimal prescription for a patient's vision

FIG. 2 shows a schematic of the components used in the method for improving accuracy of measurements from an auto-refractometer (100) to provide an optimal prescription for a patient's vision.

### DETAILED DESCRIPTION OF THE INVENTION

Following is a list of elements corresponding to a particular element referred to herein:
100 auto-refractometer
200 auto-phoropter

The present invention features a procedure to improve the accuracy of the auto-refractometer measurement by performing successive measurements where each measurement uses the previous results to compensate for a portion of the refractive error.

In order to prevent the subject to accommodate when the vision is restored or closed to be restored, it was determined that only a fraction of the refractive error measured by the previous measure should be presented to the subject. This fraction may be in the range of 5% to 80% of the measured correction, preferably 50%. Because the wavefront coming back from the eye is already partially corrected, it improves the accuracy of the successive measurements without limiting the range of measurement.

The measurement procedure may be as follows: 1) Initial measurement is taken, starting with 0 diopter correction by the auto-phoropter, represented by xD; 2) A correction is imparted to the vision by the auto-phoropter based on the measured refractive error, wherein this refractive error is used to adjust the value of xD; 3) If the subject is satisfied with the correction and have regained satisfactory visual acuity upon showing the patient a visual acuity chart of a targeted visual acuity, mostly but not limited to 20/20, stop the measurement; 4) If the subject is not satisfied with the correction, or has not regained satisfactory visual acuity, a fractional correction imparted to the vision is applied by the auto-phoropter, represented by xD*y (y being a number between 0 and 1, preferably 0.5); 5) Second measurement is taken by the auto-refractometer to update the value of xD; 6) If the subject is still unsatisfied, go back to point 4.

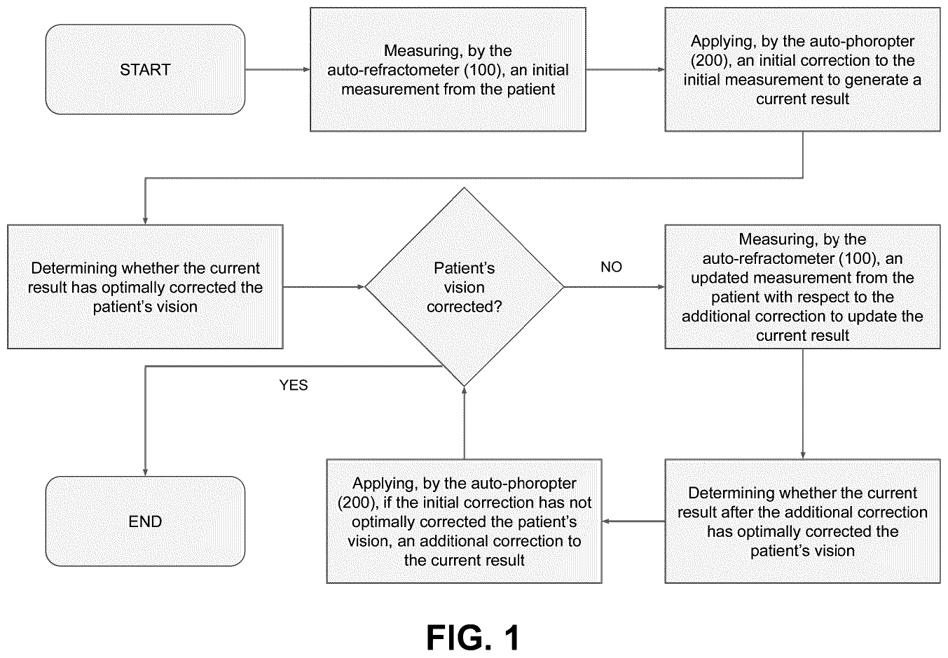

Referring now to FIG. 1, the present invention features a method for improving accuracy of measurements from an auto-refractometer (100) to provide an optimal prescription for a patient's vision. In some embodiments, the method may comprise measuring along a line of sight, by the auto-refractometer (100), an initial measurement of the patient's prescription. The initial measurement may comprise 0 diopter correction by an auto-phoropter operatively coupled to the auto-refractometer (100). The method may further comprise applying, by the auto-phoropter (200), an initial correction along the line of sight to generate an initial result, determining whether the initial correction has optimally corrected the patient's vision, and applying, by the auto-phoropter (200), if the initial correction has not optimally corrected the patient's vision, an updated fractional correction along the line of sight to replace the previous correction. The updated fractional correction may comprise the product of the previous prescription measurement and a correction factor. The method may further comprise measuring, by the auto-refractometer (100), an updated prescription measurement along the line of sight, accounting for the updated fractional correction. The method may further comprise applying, by the auto-phoropter (200) an updated full correction along the line of sight. The updated full correction may correspond to the updated prescription measurement. The method may further comprise determining whether the updated full correction has optimally corrected the patient's vision, repeating, if the updated full correction has not optimally corrected the patient's vision, applying and checking additional corrections until the patient's vision has been optimally corrected.

The present invention features a method for improving accuracy of measurements from an auto-refractometer (100) to provide an optimal prescription for a patient's vision. The method may comprise measuring, by the auto-refractometer (100), a measurement of the patient's vision in an eye and applying, by an auto-phoropter (200) operatively coupled to the auto-refractometer (100), a correction within a line of sight between the eye and the auto-refractometer. The correction may comprise an increase in visual acuity based on a correction factor. The method may further comprise repeating these steps until the patient's vision has been optimally corrected.

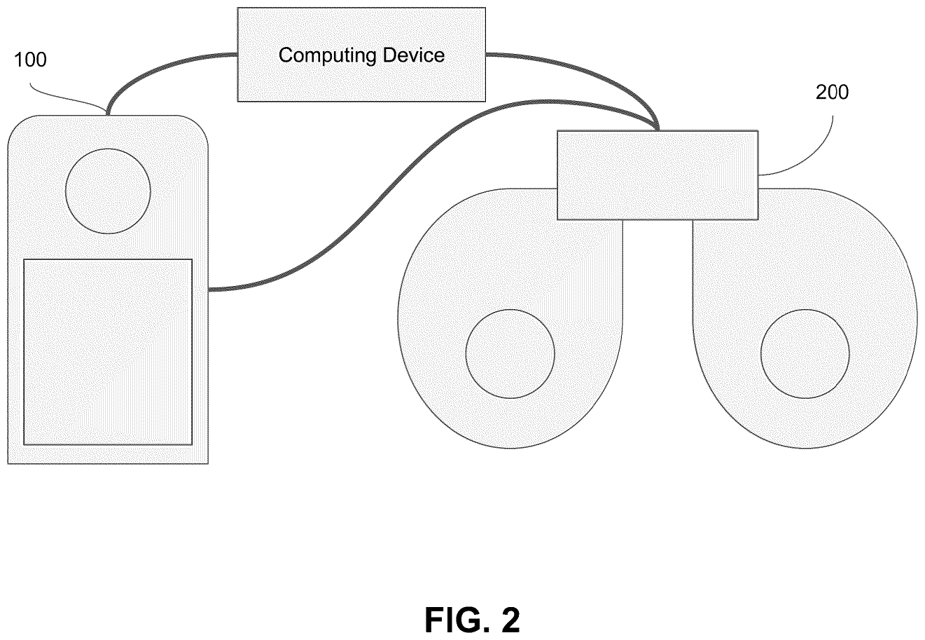

Referring now to FIG. 2, the present invention features a system for improving accuracy of measurements from an auto-refractometer (100) to provide an optimal prescription for a patient's vision. In some embodiments, the system may comprise the auto-refractometer (100), an auto-phoropter (200) operatively coupled to the auto-refractometer (100), and a computing device communicatively coupled to the auto-refractometer (100) and the auto-phoropter (200).

The computing device may comprise a processor capable of executing computer-readable instructions, and a memory component comprising a plurality of computer-readable instructions for measuring along a line of sight, by the auto-refractometer (100), an initial measurement of the patient's prescription. The initial measurement may comprise 0 diopter correction by the auto-phoropter. The computer-readable instructions may further comprise applying, by the auto-phoropter (200), an initial correction along the line of sight to generate an initial result, determining whether the initial correction has optimally corrected the patient's vision, and applying, by the auto-phoropter (200), if the initial correction has not optimally corrected the patient's vision, an updated fractional correction along the line of sight to replace the previous correction. The updated fractional correction may comprise the product of the previous prescription measurement and a correction factor. The plurality of computer-readable instructions may further comprise measuring, by the auto-refractometer (100), an updated prescription measurement along the line of sight, accounting for the updated fractional correction, and applying, by the auto-phoropter (200) an updated full correction along the line of sight. The updated full correction may correspond to the updated prescription measurement. The computer-readable instructions may further comprise determining whether the updated full correction has optimally corrected the patient's vision, and repeating, if the updated full correction has not optimally corrected the patient's vision, applying and checking additional corrections until the patient's vision has been optimally corrected.

The additional correction may comprise an increase in visual acuity based on a correction factor. In some embodiments, the correction factor may be 0.01 to 1, resulting in a 1% to 100% increase in visual acuity. In other embodiments, the correction factor may be 0.05 to 0.8, resulting in a 5% to 80% increase in visual acuity. In other embodiments, the correction factor may be 0.5, resulting in a 50% increase in visual acuity. The method may further comprise using the auto-refractometer (100) to measure an updated measurement from the patient with respect to the additional correction to update the current result. The method may further comprise determining whether the current result after the additional correction has optimally corrected the patient's vision, and if not then the method may further comprise repeating the steps of applying additional corrections and checking the patient's vision until the patient's vision has been optimally corrected. In some embodiments, determining whether the current result after the additional correction has optimally corrected the patient's vision may comprise measuring the residual refractive error after the correction, and performing a series of calculations to see if it has been diminished to zero diopters. In some embodiments, determining whether the current result after the additional correction has optimally corrected the patient's vision may comprise showing the patient a visual acuity chart and asking them to read the letters corresponding to a target visual acuity, mostly 20/20.

In some embodiments, the auto-refractometer (100) may comprise a fluidic lens. In some embodiments, the method may be used to test the eyesight of a patient, test the accuracy of a contact lens, and/or generate an eyeglass prescription for the patient.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method for improving accuracy of measurements from an auto-refractometer (100) to provide an optimal prescription for a patient's vision, the method comprising:
a. measuring along a line of sight, by the auto-refractometer (100), an initial measurement of the patient's prescription;
wherein the initial measurement comprises 0 diopter correction by an auto-phoropter operatively coupled to the auto-refractometer (100);
b. applying, by the auto-phoropter (200), an initial correction along the line of sight to generate an initial result;
c. determining whether the initial correction has optimally corrected the patient's vision;
d. applying, by the auto-phoropter (200), if the initial correction has not optimally corrected the patient's vision, an updated fractional correction along the line of sight to replace the previous correction;
wherein the updated fractional correction comprises the product of the previous prescription measurement and a correction factor;
e. measuring objectively, by the auto-refractometer (100), an updated prescription measurement along the line of sight, accounting for the updated fractional correction;
f. applying, by the auto-phoropter (200) an updated full correction along the line of sight, wherein the updated full correction corresponds to the updated prescription measurement;
g. determining whether the updated full correction has optimally corrected the patient's vision;
h. repeating, if the updated full correction has not optimally corrected the patient's vision, steps d-g until the patient's vision has been optimally corrected.

2. The method of claim 1, wherein the correction factor is 0.01 to 1, resulting in a 1% to 100% increase in visual acuity.

3. The method of claim 2, wherein the correction factor is 0.05 to 0.8, resulting in a 5% to 80% increase in visual acuity.

4. The method of claim 2, wherein the correction factor is 0.5, resulting in a 50% increase in visual acuity.

5. The method of claim 1, wherein the auto-refractometer (100) comprises a fluidic lens.

6. The method of claim 1, wherein the method is used to test accuracy of a contact lens.

7. A method for improving accuracy of measurements from an auto-refractometer (100) to provide an optimal prescription for a patient's vision, the method comprising:
a. measuring, by the auto-refractometer (100), a measurement of the patient's vision in an eye;
b. applying, by an auto-phoropter (200) operatively coupled to the auto-refractometer (100), a correction within a line of sight between the eye and the auto-refractometer;
wherein the correction comprises an increase in visual acuity based on a correction factor; and
c. repeating steps a-b until the patient's vision has been optimally corrected.

8. The method of claim 7, wherein a first measurement comprises 0 diopter correction by the auto-phoropter (200).

9. The method of claim 7, wherein the correction factor is 0.01 to 1, resulting in a 1% to 100% increase in visual acuity.

10. The method of claim 9, wherein the correction factor is 0.05 to 0.8, resulting in a 5% to 80% increase in visual acuity.

11. The method of claim 9, wherein the correction factor is 0.5, resulting in a 50% increase in visual acuity.

12. The method of claim 7, wherein the auto-refractometer (100) comprises a fluidic lens.

13. The method of claim 7, wherein the method is used to test accuracy of a contact lens.

14. A system for improving accuracy of measurements from an auto-refractometer (100) to provide an optimal prescription for a patient's vision, the system comprising:
a. the auto-refractometer (100);
b. an auto-phoropter (200) operatively coupled to the auto-refractometer (100); and
c. a computing device communicatively coupled to the auto-refractometer (100) and the auto-phoropter (200), comprising a processor capable of executing computer-readable instructions, and a memory component comprising a plurality of computer-readable instructions for:
i. measuring along a line of sight, by the auto-refractometer (100), an initial measurement of the patient's prescription;
wherein the initial measurement comprises 0 diopter correction by the auto-phoropter;
ii. applying, by the auto-phoropter (200), an initial correction along the line of sight to generate an initial result;
iii. determining whether the initial correction has optimally corrected the patient's vision;
iv. applying, by the auto-phoropter (200), if the initial correction has not optimally corrected the patient's vision, an updated fractional correction along the line of sight to replace the previous correction;
wherein the updated fractional correction comprises the product of the previous prescription measurement and a correction factor;
v. measuring, by the auto-refractometer (100), an updated prescription measurement along the line of sight, accounting for the updated fractional correction;
vi. applying, by the auto-phoropter (200) an updated full correction along the line of sight, wherein the updated full correction corresponds to the updated prescription measurement;
vii. determining whether the updated full correction has optimally corrected the patient's vision;
viii. repeating, if the updated full correction has not optimally corrected the patient's vision, steps iv-vii until the patient's vision has been optimally corrected.

15. The system of claim 14, wherein the correction factor is 0.01 to 1, resulting in a 1% to 100% increase in visual acuity.

16. The system of claim 15, wherein the correction factor is 0.05 to 0.8, resulting in a 5% to 80% increase in visual acuity.

17. The system of claim 15, wherein the correction factor is 0.5, resulting in a 50% increase in visual acuity.

18. The system of claim 14, wherein the auto-refractometer (100) comprises a fluidic lens.

19. The system of claim 14, wherein the method is used to test accuracy of a contact lens.
